(19) United States
(12) Patent Application Publication
(10) Pub. No.: US 2021/0033562 A1
(43) Pub. Date: Feb. 4, 2021

(54) METHOD FOR CALIBRATING AN ANALYTICAL MEASURING DEVICE AND MEASURING POINT FOR ANALYZING A PROCESS MEDIUM AND FOR CALIBRATING AN ANALYTICAL MEASURING DEVICE

(71) Applicant: Endress+Hauser Conducta GmbH+Co. KG, Gerlingen (DE)

(72) Inventors: Torsten Pechstein, Radebeul (DE); Michael Hanko, Dresden (DE); Dagmar Kaschuba, Gerlingen (DE); Erik Hennings, Freiberg (DE); Thomas Pfauch, Leipzig (DE); Stephan Jugert, Dresden (DE); Jens Vettermann, Großweitzschen (DE)

(21) Appl. No.: 16/942,436

(22) Filed: Jul. 29, 2020

(30) Foreign Application Priority Data
Jul. 29, 2019 (DE) 10 2019 120 420.8

(51) Int. Cl. G01N 27/419; G01N 27/08

(52) U.S. Cl. CPC G01N 27/419; G01N 27/4162; G01N 27/08

(57) ABSTRACT

The present disclosure relates to a method for calibrating an analytical measuring device in a measuring point, wherein the method comprises at least the following steps: closing the inlet valve so that no process medium is fed from the first inlet into the measuring point, emptying the measuring point of the process medium through the outlet valve, closing the outlet valve, feeding a predetermined volume of the calibration medium into the measuring point through the inlet valve from the second inlet, circulating the calibration medium through the pump so that the flow circuit is generated and the calibration medium flows against the analytical measuring device, wherein a predetermined flow velocity of the calibration medium is adjusted by the pump, and calibrating the analytical measuring device.

### CROSS-REFERENCE TO RELATED APPLICATION

The present application is related to and claims the priority benefit of German Patent Application No. 10 2019 120 420.8, filed on Jul. 29, 2019, the entire contents of which are incorporated herein by reference.

### TECHNICAL FIELD

The present disclosure relates to various methods for calibrating an analytical measuring device and to a measuring point for analyzing a process medium and for calibrating an analytical measuring device.

### BACKGROUND

In analytical measurement technology, especially in the fields of water management and environmental analysis and in industry, for example in food technology, biotechnology and pharmaceuticals, as well as for various laboratory applications, measurands such as pH value, conductivity or the concentration of analytes such as ions or dissolved gases in a gaseous or liquid measurement medium are vitally important. These measurands can be detected and/or monitored, for example, by means of analytical measuring devices, especially electrochemical sensors, such as potentiometric, amperometric, voltammetric or coulometric sensors, or else conductivity sensors.

In the field of water management, especially in the monitoring of drinking water, ballast water in ships, water in swimming pools, so-called disinfection sensors are used, which are suitable for measuring different parameters, e.g. chlorine, chlorine dioxide, bromine, hydrogen peroxide, etc. Such sensors are used when the content of the respective species has to be monitored in order to ensure an antibacterial state of the process systems.

Disinfection sensors also show a dependence of the measured value on the inflow of the sensor membrane. For reliable measurement results, it is therefore important to know the inflow and to be able to set it precisely. Disinfection sensors are usually part of a measuring point or even of a control loop. Measuring points can be designed, for example, as flow fittings or as screw-in nozzles. Flow fittings are preferred over screw-in nozzles, since the flow at the sensor membrane can be set with these.

Disinfection sensors usually operate according to an electrochemical measuring principle. By electrochemical reaction, temperature influences, or by the chemical process conditions themselves, the sensor experiences a shift of the measurement signal which can be reflected in a changed sensor characteristic.

To ensure sufficient measurement accuracy, it is necessary to calibrate the sensor and adjust the zero point and/or slope.

Conventional disinfection sensors are removed from the fitting for calibration and, in a separate vessel, reference materials for the zero point and/or slope determination are applied. Another possibility of calibration consists of sampling at the fitting and measuring this sample via a reference measurement method; in the case of chlorine, with the so-called colorimetric DPD test. As a result, an offset or slope correction of the disinfection sensor is only possible with considerable effort.

However, the DPD test method has a more than negligible measurement error which can be transmitted to the disinfection sensor during its adjustment.

A plurality of sometimes complex work steps are thus required for the calibration of a disinfection sensor. These work steps represent a risk of error for an incorrect adjustment of the disinfection sensor, which might not be detected by the user during operation of the disinfection sensor.

Moreover, the previously known calibration method has the disadvantage that during calibration, the disinfection sensor is usually exposed to other flow conditions, other temperatures, other compositions of the water matrix with respect to the reference solution than in the process flow fitting, as a result of which measurement errors can be generated.

The removal of the disinfection sensor from the fitting represents an additional effort for the operator of the measuring point.

### SUMMARY

It is therefore an object of the present disclosure to provide a calibration method which avoids the aforementioned disadvantages.

This object is achieved by a method for calibrating an analytical measuring device in a measuring point according to claim 1.

The method according to the present disclosure comprises at least the following steps:
Providing a measuring point through which a process medium flows and an analytical measuring device, wherein the measuring point has an inlet valve, an outlet valve, an analysis container, a dosing container and a pump,

wherein the inlet valve is connected to a first inlet for feeding in the process medium, a second inlet for feeding in a calibration medium, the analysis container and the dosing container,

wherein the outlet valve is connected to an outlet, the analysis container and the dosing container,

wherein the inlet valve, the analysis container, the dosing container and the outlet valve are connected to one another in such a way that a flow circuit can be realized in the measuring point,

wherein the pump is arranged in such a way that it is suitable for generating the flow circuit,

wherein the analytical measuring device is arranged in the analysis container and is in contact with the process medium,
Closing the inlet valve 10 so that no process medium is fed from the first inlet 3 into the measuring point 1,
Emptying the measuring point of the process medium through the outlet valve,
Closing the outlet valve,
Feeding a predetermined volume of the calibration medium into the measuring point through the inlet valve from the second inlet,
Circulating the calibration medium through the pump so that the flow circuit is generated and the calibration medium flows against the analytical measuring device, wherein a predetermined flow velocity of the calibration medium is adjusted by the pump,
Calibrating the analytical measuring device.

The inventive method for calibrating an analytical measuring device enables particularly precise calibration of an analytical measuring device in the process installation state.

According to one embodiment of the present disclosure, before the step of closing the inlet valve, a step of measuring the process medium by the analytical measuring device and a step of measuring the flow velocity of the process medium is carried out by a flow meter, wherein, during the step of circulating the calibration medium by the pump, the predetermined flow velocity of the calibration medium is adjusted such that the flow velocity of the calibration medium corresponds to the measured flow velocity of the process medium.

The inventive method for calibrating an analytical measuring device enables particularly precise calibration of an analytical measuring device in the process installation state. Since the same flow velocity of the process medium as in measuring operation is selected during the calibration of the analytical measuring device, a very precise calibration is possible.

The above-mentioned object is achieved by a method for calibrating an analytical measuring device in a measuring point according to claim 3.

The method according to the present disclosure comprises at least the following steps:
Providing a measuring point through which a process medium flows and an analytical measuring device, wherein the measuring point has an inlet valve, an analysis container, a dosing container and a pump,

wherein the inlet valve is connected to a first inlet for feeding in a process medium, a second inlet for feeding in a calibration medium and the analysis container,

wherein the pump is arranged in such a way that it is suitable for generating a predetermined flow through the measuring point,

wherein the analytical measuring device is arranged in the analysis container and is in contact with the process medium,
Closing the inlet valve 10 so that no process medium is fed from the first inlet 3 into the measuring point 1,
Emptying the measuring point of the process medium,
Feeding the calibration medium into the measuring point through the inlet valve from the second inlet to the outlet so that the flow is generated and calibration medium flow against the analytical measuring device,

wherein a predetermined flow velocity of the calibration medium is set by the pump,
Calibrating the analytical measuring device.

The inventive method for calibrating an analytical measuring device enables particularly precise calibration of an analytical measuring device in the process installation state. Since the calibration medium used for calibration of the analytical measuring device flows directly to the outlet, only a few components are necessary for implementing the method.

In one embodiment of the present disclosure, the analytical measuring device has a cross-sensitivity to the calibration medium, and wherein the step of calibrating the analytical measuring device is a calibration based on the cross-sensitivity to the calibration medium. It is thus possible to calibrate different sensors simultaneously by means of a calibration medium.

In one embodiment of the present disclosure, the calibration medium contains hypochlorous acid. Thus, the calibration is particularly simple.

In one embodiment of the present disclosure, the calibration medium comprises a pH buffer for the calibration of a pH sensor. Thus, the calibration is particularly simple.

In one embodiment of the present disclosure, the calibration medium comprises a pH buffer and a salt of hypochlorous acid, wherein the step of feeding the predetermined volume of the calibration medium to the measuring point comprises separate feeding of the pH buffer and the salt of the hypochlorous acid. As a result, the calibration medium is only generated in situ and at the point in time when it is needed, whereby the autonomy duration of the calibration method is increased. The pH buffer as well as the salt of the hypochlorous acid are separately more durable than when mixed with the calibration medium.

In one embodiment of the present disclosure, the calibration medium comprises demineralized water and a stock solution having a known concentration of an analyte. As a result, the calibration medium is only generated in situ and at the point in time when it is needed, whereby the autonomy duration of the calibration method is increased.

The aforementioned object is further achieved by a measuring point for analyzing a process medium and for calibrating an analytical measuring device as claimed in claim 9.

The measuring point according to the present disclosure comprises:

an inlet valve, an outlet valve, an analysis container, a dosing container, and a pump with a regulatable delivery rate,

wherein the inlet valve is connected to a first inlet for feeding in a process medium, a second inlet for feeding in a calibration medium, the analysis container and the dosing container,

wherein the outlet valve is connected to an outlet, the analysis container and the dosing container,

wherein the inlet valve, the analysis container, the dosing container and the outlet valve are connected to one another in such a way that a flow circuit can be realized in the measuring point,

wherein the pump is arranged in such a way that it is suitable for generating the flow circuit,

wherein the analytical measuring device is arranged in the analysis container in such a way that the flow circuit can flow into the analytical measuring device.

According to one embodiment of the present disclosure, the measuring point further comprises a bypass channel which connects the first inlet and the outlet in order to guide a part of the process medium from the first inlet past the analysis container and the dosing container to the outlet, wherein a first drive means of the pump is arranged in the bypass channel and a second drive means of the pump is arranged in the flow circuit, wherein the first drive means is suitable for driving the second drive means. The measuring point is thus suitable for driving the first drive means currentlessly via the second drive means. The measuring point is thus also suitable, by means of the first drive means and the second drive means, for representing the flow velocity prevailing in the bypass channel on the flow circuit in the measuring point.

The above-mentioned object is also achieved by a measuring point for calibrating an analytical measuring device as claimed in claim 11.

The measuring point according to the present disclosure comprises:

an inlet valve, an analysis container and a pump with an adjustable delivery rate,

wherein the inlet valve is connected to a first inlet for feeding in a process medium, a second inlet for feeding in a calibration medium, the analysis container and the dosing container,

wherein the inlet valve and the analysis container are connected to one another in such a way that a flow circuit can be realized in the measuring point,

wherein the pump is arranged in such a way that it is suitable for generating the flow circuit,

wherein the analytical measuring device is arranged in the analysis container in such a way that the flow can flow against the analytical measuring device.

Such a measuring point has a minimum number of components.

According to one embodiment of the present disclosure, the inlet valve is configured as a multi-way valve.

According to one embodiment of the present disclosure, the analytical measuring device is a chlorine sensor and/or a chlorine dioxide sensor and/or a bromine sensor and/or a pH sensor and/or a conductivity sensor and/or a dissolved oxygen sensor.

### BRIEF DESCRIPTION OF THE DRAWINGS

The present disclosure will be explained in more detail on the basis of the following description of the figure. They show:

FIG. 1: a schematic depiction of a measuring point according to the present disclosure,

FIG. 2: a schematic depiction of an embodiment of the measuring point from FIG. 1 with a bypass channel,

FIG. 3: a schematic depiction of an embodiment of the measuring point from FIG. 1 with open outlet.

### DETAILED DESCRIPTION

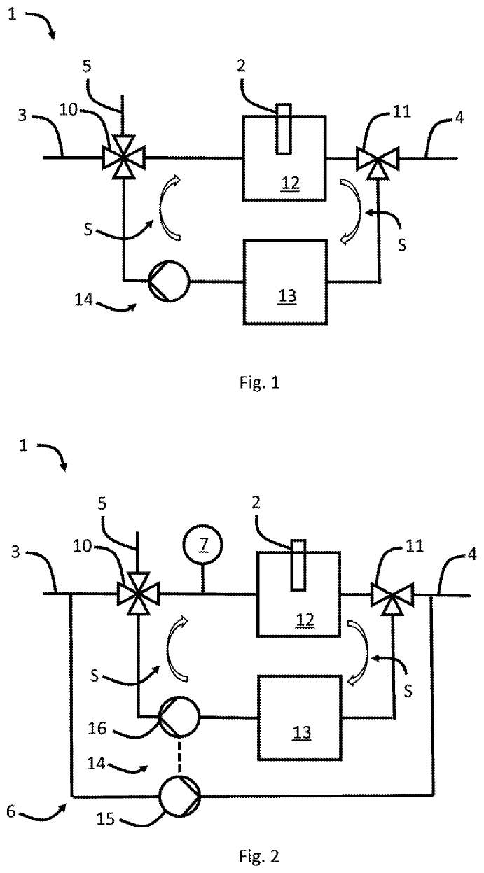

FIG. 1 shows a schematic depiction of a measuring point 1 according to the present disclosure. According to one embodiment, the measuring point 1 is a flow-through measuring point. The measuring point 1 comprises an inlet valve 10, an outlet valve 11, an analysis container 12, a dosing container 13 and a pump 14. An analytical measuring device 2 is arranged in the analysis container 12.

The inlet valve 10 is connected to a first inlet 3 for feeding in a process medium, a second inlet 5 for feeding in a calibration medium, the analysis container 12 and the dosing container 13.

The outlet valve 11 is connected to an outlet 4, the analysis container 12 and the dosing container 13. The inlet valve 10 is preferably configured as a multi-way valve, for example as a four-way valve. In one embodiment, the inlet valve 10 can be designed in such a way that the four paths of the inlet valve 10 are arranged in a manner spatially separated.

The inlet valve 10, the analysis container 12, the dosing container 13 and the outlet valve 11 are connected to one another in such a way that a flow circuit S can be realized in the measuring point 1. The pump 14 is arranged in such a way that it is suitable for generating the flow circuit S. In FIG. 1, the pump 14 is arranged between the inlet valve 10 and the dosing container 13. However, the pump 14 can also be arranged at other points within the flow circuit S. The pump 14 has a regulatable delivery rate. The analytical measuring device 2 is arranged in the analysis container 12 in such a way that the flow circuit S can flow into the analytical measuring device 2.

The analytical measuring device 2 is, for example, a chlorine sensor and/or a chlorine dioxide sensor and/or a bromine sensor and/or a pH sensor and/or a conductivity sensor and/or a dissolved oxygen sensor.

FIG. 2 shows a second embodiment of the measuring point 1 with a so-called bypass channel 6. The bypass channel 6 connects the first inlet 3 and the outlet 4, in order to guide the process medium from the first inlet 3 to the outlet 4. The bypass channel 6 enables a portion of the process medium to be guided from the first inlet 3 past the analysis container 12 and the dosing container 13 directly to the outlet 4. A first drive means 15 of the pump 14 is arranged in the bypass channel 6 and a second drive means 16 of the pump is arranged in the flow circuit S. The first and second drive means 15, 16 is, for example, a paddle wheel or a turbine type. The first drive means 15 is suitable for driving the second drive means 16. The first drive means 15 is connected to the second drive means 16 via a drive shaft, for example. A transmission means, for example a transmission, can also be arranged between the first drive means 15 and the second drive means 16, in order to achieve different rotational speeds of the two drive means 15, 16.

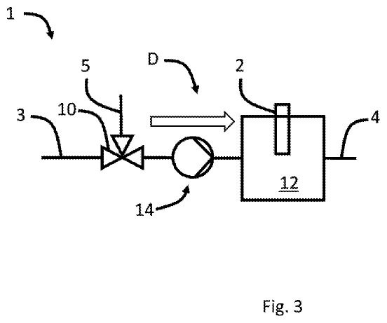

FIG. 2 further shows a flow meter 7 arranged between the inlet valve 10 and the analysis container 12. The flow meter 7 can, of course, be arranged at other positions in the flow circuit S. The flow meter 7 enables a flow velocity to be measured. Of course, the flow meter 7 can also be used in the measuring points 1 depicted in FIG. 1 or FIG. 3 (not shown there). Alternatively or additionally, the pump 14 can be used to measure the flow velocity.

FIG. 3 shows an alternative third embodiment of the measuring point 1 with a so-called open outlet 4. This embodiment differs from the previously detailed embodiments in that no dosing container 13 is present. Thus, this embodiment is not suitable for designing a flow circuit S in the measuring point 1. However, the pump 14 is suitable for forming a flow D in the measuring point 1. The analytical measuring device 2 is arranged in the analysis container 12 in such a way that the flow S can flow against the analytical measuring device 2.

The third embodiment shown in FIG. 3 is suitable, like the second embodiment shown in FIG. 2, to be provided with a bypass channel to drive the pump 14 (not shown).

In the following, the method for calibrating the analytical measuring device 2 will be described.

In a first step, the measuring point 1 described above with reference to FIG. 1 is provided.

The measuring point 1 is provided in such a way that the process medium flows through the measuring point 1. In other words, the measuring point 1 is in operation. The process medium thus flows from the first inlet 3 through the measuring point 1 to the outlet 4.

The process medium is guided from the first inlet 3 through the analysis container 12 to the outlet 4. In this case, the inlet valve 10 is switched in such a way that the inlet valve 10 communicates with the first inlet 3 and the analysis container 12, and the outlet valve 11 is switched in such a way that the outlet valve 11 communicates only with the analysis container 12 and the outlet 4.

In a next step, the inlet valve 10 is closed so that no additional process medium is fed from the first inlet 3 into the measuring point 1. The outlet valve 11 remains open. The outlet valve 11 remains open until the measuring point 1 is emptied of the process medium. Then the outlet valve 11 is closed.

Optionally, before the step of closing the inlet valve 10, a step can be performed of measuring the process medium using the analytical measuring device 2 and a step of measuring the flow velocity of the process medium using a flow meter 7.

In a following step, a predetermined volume of a calibration medium is fed into the measuring point 1 through the second inlet 5 of the inlet valve 10.

Optionally, a step of flushing the measuring point 1 can take place before the step of feeding in a calibration medium. During flushing, for example, water or another flushing agent is conducted from the second inlet 5 of the inlet valve 10 through the analysis container 12, optionally the dosing container 13, the pump 14 and the outlet valve 11 to the outlet 4. The flushing prepares the measuring point 1 for the subsequent calibration, which contributes to an accurate calibration. Alternatively, the flushing step, when analyte-free water is used, may serve to determine the zero point of the analytical measuring device 2.

A hypochlorous acid is used as the calibration medium for disinfection sensors, for example. Alternatively, the calibration medium comprises demineralized water and a stock solution having a known concentration of an analyte. Alternatively, the calibration medium comprises a pH buffer for calibrating a pH sensor as the analytical measuring device 2.

Alternatively, the calibration medium comprises a pH buffer and a salt of hypochlorous acid. In this alternative, the feed step comprises feeding the pH buffer separate from feeding the salt of the hypochlorous acid. Separately here means that the pH buffer and the salt of the hypochlorous acid are fed into the measuring point 1 or combined separately in time. Alternatively, the pH buffer and the salt of the hypochlorous acid could also be combined shortly before feeding into the measuring point 1 from locally separate containers and thus fed into the measuring point 1 at the same time. The advantage of a time-separated feeding or local separation and combining only shortly before feeding into the measuring point 1 is that the calibration means provided in this way has significantly longer durability than the combined calibrating means or known calibrating means, such as hypochlorous acid.

The calibration medium and process medium are then circulated in the measuring point 1 by the pump 14 so that the flow circuit S is generated, and the calibration medium flows against the analytical measuring device 2.

The pump 14 controls the flow circuit S in such a way that a predetermined flow velocity of the calibration medium/process medium mixture is achieved.

The predetermined flow velocity of the calibration medium/process medium mixture is preferably set in such a way that the flow velocity of the calibration medium/process medium mixture corresponds to the flow velocity of the process medium measured by the flow meter in measuring mode. Accurate calibration is thus possible, since the operating conditions of the analytical sensor 2, i.e. the exact flow velocity, in measuring mode are also taken into account during the calibration operation.

As indicated in FIG. 1 by the arrows, the flow circuit S produced by the pump 14 runs in the same direction as the flow direction of the process medium in measuring mode. The flow circuit S runs from the inlet valve 10, via the analysis container 12, via the outlet valve 11, via the dosing tank 13, via the pump 14 to the analysis container 12. The inlet valve 10 and the outlet valve 11 are opened in such a way that the analysis container 12 and the dosing container 13 communicate with each other in fluid communication.

In a further step, the analytical measuring device 2 is calibrated. When calibrating the analytical measuring device, for example the slope or zero point of the analytical measuring device 2 is adjusted.

In the case of a hypochlorous acid as the calibration medium, the slope of the analytical measuring device 2 is adjusted during the calibration step.

In the case of a pH buffer as the calibration medium, the zero point or slope of the pH sensor is calibrated as the analytical measuring device 2 during the calibration step.

In the case of the pH buffer and a salt of a hypochlorous acid as the calibration medium, the slope of the analytical measuring device 2 is adjusted during the calibration step.

In the case of demineralized water and a stock solution having a known concentration of an analyte as a calibration medium, the slope of the analytical measuring device 2 is adjusted during the calibration step.

In one embodiment of the calibration step, the calibration is based on a cross-sensitivity of the analytical measuring device 2 to the calibration medium. Of course, an analytical measuring device 2 which has this cross-sensitivity to the calibration medium is calibrated.

FIG. 2 shows a variant of the calibration method described with reference to FIG. 1. In this case, the first drive means 15 of the pump 14 is driven by the second drive means 16 of the pump 14 during the step of circulating the calibration medium. The flow velocity of the calibration medium in the flow circuit S is set here by setting a transmission ratio of the first drive means 15 and second drive means 16 which are mechanically connected to one another. In this variant, the flow meter 7 is used to check the flow velocity in the flow circuit S.

FIG. 3 shows an alternative second calibration method of the analytical measuring device 2. This second calibration method differs from the previously mentioned calibration methods in that the calibration medium is not pumped in the circuit, but rather, flows from the second inlet 5 via the pump 14, the analysis container 12 to the outlet 4 in order to form a flow D.

This alternative second calibration method is of course compatible with the type of drive of the pump 14 described with reference to FIG. 2 by means of a bypass channel.

Further advantages of the calibration method described are that a faster and more accurate measurement is possible in the event of burst disinfections.

The calibration method also makes it possible to dispense with conventional calibration of the analytical measuring device 2 by means of a so-called cuvette test or colorimetric DPD test by mixing with a defined pH and a defined amount of salt of the hypochlorous acid.

1. A method for calibrating an analytical measuring device in a measuring point, the method comprising:
providing a measuring point through which a process medium flows and an analytical measuring device, wherein the measuring point has an inlet valve, an outlet valve, an analysis container, a dosing container and a pump, wherein the inlet valve is connected to a first inlet, for feeding in the process medium, a second inlet for feeding in a calibration medium, the analysis container, and the dosing container, wherein the outlet valve is connected to an outlet, the analysis container, and the dosing container, wherein the inlet valve, the analysis container, the dosing container, and the outlet valve are connected to one another such that a flow circuit can be realized in the measuring point, wherein the pump is arranged in such a way that it is suitable for generating the flow circuit, and wherein the analytical measuring device is arranged in the analysis container and is in contact with the process medium;
closing the inlet valve so that no process medium is fed from the first inlet into the measuring point;
emptying the measuring point of the process medium through the outlet valve;
closing the outlet valve;
feeding a predetermined volume of the calibration medium into the measuring point through the inlet valve from the second inlet;
circulating the calibration medium through the pump so that the flow circuit is generated and calibration medium flows against the analytical measuring device, wherein a predetermined flow velocity of the calibration medium is adjusted by the pump; and
calibrating the analytical measuring device.

2. The method according to claim 1, wherein before the step of closing the inlet valve, a step is performed of measuring the process medium using the analytical measuring device and a step of measuring the flow velocity of the process medium using a flow meter, wherein in the step of circulating the calibration medium by the pump, the predetermined flow velocity of the calibration medium is set such that the flow velocity of the calibration medium corresponds to the measured flow velocity of the process medium.

3. A method for calibrating an analytical measuring device in a measuring point, the method comprising:
providing a measuring point through which a process medium flows and an analytical measuring device, wherein the measuring point has an inlet valve, an analysis container and a pump, wherein the inlet valve is connected to a first inlet for feeding in a process medium, a second inlet for feeding in a calibration medium, and the analysis container, wherein the pump is arranged in such a way that it is suitable for generating a predetermined flow through the measuring point, and wherein the analytical measuring device is arranged in the analysis container and is in contact with the process medium;
closing the inlet valve so that no process medium is fed from the first inlet into the measuring point;
emptying the measuring point of the process medium;
feeding the calibration medium into the measuring point through the inlet valve from the second inlet to the outlet so that the flow is generated and the calibration medium flows against the analytical measuring device, wherein a predetermined flow velocity of the calibration medium is set by the pump; and
calibrating the analytical measuring device.

4. The method according to claim 3, wherein the analytical measuring device has a cross-sensitivity to the calibration medium, and the step of calibrating the analytical measuring device is a calibration which is based on the cross-sensitivity to the calibration medium.

5. The method according to claim 3, wherein the calibration medium contains hypochlorous acid.

6. The method according to claim 3, wherein the calibration medium comprises a pH buffer for the calibration of a pH sensor.

7. The method according to claim 3, wherein the calibration medium comprises a pH buffer and a salt of hypochlorous acid, and wherein the step of feeding the predetermined volume of the calibration medium to the measuring point comprises separately feeding the pH buffer and the salt of the hypochlorous acid.

8. The method according to claim 3, wherein the calibration medium comprises demineralized water and a stock solution having a known concentration of an analyte.

9. A measuring point for analyzing a process medium and for calibrating an analytical measuring device, comprising:
an inlet valve, an outlet valve, an analysis container, a dosing container and a pump with a regulatable delivery rate,
wherein the inlet valve is connected to a first inlet for feeding in a process medium, a second inlet for feeding in a calibration medium, the analysis container, and the dosing container,
wherein the outlet valve is connected to an outlet, the analysis container and the dosing container,
wherein the inlet valve, the analysis container, the dosing container and the outlet valve are connected to one another in such a way that a flow circuit can be realized in the measuring point,
wherein the pump is arranged in such a way that it is suitable for generating the flow circuit, and
wherein the analytical measuring device is arranged in the analysis container in such a way that the flow circuit can flow into the analytical measuring device.

10. The measuring point according to claim 9, wherein the measuring point further comprises a bypass channel which connects the first inlet and the outlet in order to guide a part of the process medium from the first inlet past the analysis container and the dosing container to the outlet,
wherein a first drive means of the pump is arranged in the bypass channel, and a second drive means of the pump is arranged in the flow circuit, wherein the first drive means is suitable for driving the second drive means.

11. A measuring point for analyzing a process medium and for calibrating an analytical measuring device, comprising:
an inlet valve, an analysis container and a pump with an adjustable delivery rate,
wherein the inlet valve is connected to a first inlet for feeding in a process medium, a second inlet for feeding in a calibration medium and the analysis container,
wherein the inlet valve and the analysis container are connected to one another such that a flow in the measuring point can be realized,
wherein the pump is arranged such that it is suitable for generating the flow,
wherein the analytical measuring device is arranged in the analysis container such that the flow can flow against the analytical measuring device.

12. The measuring point according to claim 11, wherein the inlet valve is configured as a multiway valve.

13. The measuring point according to claim 11, wherein the analytical measuring device is an electrochemical chlorine sensor and/or an electrochemical chlorine dioxide sensor and/or a bromine sensor and/or a pH sensor and/or a conductivity sensor and/or a dissolved oxygen sensor.
